(19) United States
(12) Patent Application Publication
(10) Pub. No.: US 2019/0247030 A1
(43) Pub. Date: Aug. 15, 2019

(54) System and Method for Collecting, Enriching and Isolating Trophoblast Cells From Endocervical Canal

(71) Applicant: TrophoDiagnostics, LLC, Agoura Hills, CA (US)

(72) Inventors: Zitao Liu, Canaan, CT (US); Lian Liu, Agoura Hills, CA (US)

(73) Assignee: TrophoDiagnostics, LLC, Agoura Hills, CA (US)

(21) Appl. No.: 15/894,949

(22) Filed: Feb. 13, 2018

(51) Int. Cl. A61B 10/02; G01N 33/569; C07K 16/28; G01N 1/40; G01N 1/34; C12Q 1/6883; A61B 5/00

(52) U.S. Cl. CPC A61B 10/0291; G01N 33/56966; C07K 16/2833; C12Q 2600/156; G01N 1/34; C12Q 1/6883; A61B 5/4362; G01N 1/405

(57) ABSTRACT

Improved systems and methods for collecting, isolating and analyzing trophoblasts from the endocervical canal for prenatal genetic testing are disclosed. Particularly, an endocervical sample collection device is disclosed, where that collection device is enhanced with surface nanostructures/microstructures and functionalized with trophoblast specific antibodies to allow the collection device to preferentially adhere to and pick up fetal trophoblasts as opposed to maternal cells and/or non-cellular materials such as endocervical mucus. In addition, a non-caustic mucolytic preparation for treating endocervical sample to remove mucus while keeping the trophoblast cells intact and viable for downstream culturing and processing is disclosed.

### PRIORITY APPLICATION

This application claims the benefit of U.S. Provisional Application 62/458,375, filed on Feb. 13, 2017, which is incorporated herein by reference in its entirety.

### FIELD OF THE INVENTION

This invention relates generally to systems and methods for collecting fetal trophoblasts from endocervical canal for prenatal genetic testing and, and in particular, to a endocervical sample collection device with nanostructured and/or microstructured sample collection surface with nanoscale and/or microscale topography, and to a composition of matter for removing mucus from the sample collected from the endocervical canal using the collection device while minimizing damaging or killing the trophoblast cells.

### BACKGROUND OF THE INVENTION

Every year, 8 million children worldwide are born with genetic birth defects (accounts for 6% of global births), 3 million children die of genetic birth defects by age 5, and almost as many are permanently affected by their birth defects. Prenatal genetic testing can help to reduce the number of children born with genetic birth defects by allowing parents-to-be to know early on whether the fetus has genetic abnormality that increases the risk for birth defect.

The current standard prenatal genetic tests are chorionic villus sampling (CVS) and amniocentesis. CVS is performed at 10 to 14 weeks of pregnancy by inserting a needle through either the mother's abdomen or vagina to withdraw a sample of chorionic villi from the placenta where it attaches to the wall of the uterus. Amniocentesis is performed at 15 to 20 weeks of pregnancy by inserting a thin needle into the mother's abdomen to extract a sample of amniotic fluid surrounding the fetus. Both CVS and amniocentesis are invasive and carry significant risk for miscarriage, which are 1% and 0.25% for CVS and amniocentesis, respectively.

Non-Invasive Prenatal Testing (NIPT) is now emerging as another option for prenatal genetic testing. Current NIPT are performed at 10 to 22 weeks of pregnancy and is carried out by detecting and analyzing fetal DNA fragments circulating (called cell-free fetal DNA) in maternal blood. Although it is far less invasive than CVS or amniocentesis, drawing maternal blood is still required. So far, cell-free fetal DNA based NIPT is mostly limited to detecting chromosomal aneuploidies such as Down Syndrome, it is not capable of detecting mosaicism or single point chromosomal mutation.

Recently, a new form of NIPT based on fetal trophoblasts harvested from endocervical canal has been developed for fetal genetic testing. Trophoblasts are fetal cells that form the outer layer of blastocyst, which function to attach embryo to maternal uterine wall and later develop into a large part of placenta to supply nutrition to the embryo and the fetus. After 5 weeks of gestational age, trophoblasts shed from the regressing chorionic villi into the cervical canal and accumulate behind the cervical mucus at the level of the internal os. The endocervical trophoblast based NIPT test can be carried out at 5 to 20 weeks of gestational age, significantly earlier than other prenatal genetic diagnostic tests. Since the new NIPT is based on whole fetal cells rather than cell free fetal DNA fragments, they have the potential of being used for a wider range of fetal genetic testing, including aneuploidy, mosaicism, Rh fetal status in Rh negative mother, mitochondrial DNA disease, and single point chromosome mutation such as cystic fibrosis and hemoglobinopathies.

Despite the promise of endocervical trophoblast based NIPT, there remain various challenges. One challenge is that the amount of trophoblast cells present in the endocervical canal is very small. It has been estimated that the ratio of fetal trophoblast cells to maternal cells in the endocervical canal is approximately 1 in 2000. Another challenge is that the trophoblasts collected from the endocervical canal are often immersed in thick layers endocervical mucus, which is a highly oligomerized polymer composed of water and various macromolecular glycoproteins. Removing the mucus without damaging or reducing the number of intact trophoblasts cells for prenatal genetic testing is difficult. Therefore, there is a need for systems and methods for effective collection, enrichment, and isolation of trophoblasts from the endocervical canal for prenatal genetic testing.

There is also a need for a trophoblast collection device that is less abrasive and safer for the patient. In the past, cotton swabs, aspiration catheter, endometrial biopsy, endocervical canal lavage, wooden and plastic spatulas, and cervical collection brushes have been used to collect fetal trophoblasts from endocervical canal with variable success rates, among them, cytobrush is the most effective, with a success rate as high as 95% for capturing endocervical trophoblasts. However, the nylon bristles of the cytobrush is abrasive and can cause endocervical bleeding. Patients undergoing trophoblasts harvesting for fetal genetic testing are typically in their early pregnancy, they may become alarmed and worried when they spot vaginal bleeding. In vulnerable patients, the bleeding or bruising may cause infection and miscarriage. Furthermore, the current cytobrush does not have the ability to selectively pick up fetal trophoblast cells over maternal cells or non-cellular materials such as endocervical mucus.

There is also a need for a endocervical sample treatment solution that will release the trophoblast cells from the mucus while keeping the cell membrane intact and/or cell viable for further testing. Currently, after an endocervical sample is collected, it is typically treated with a fixative solution to prevent it from degradation, autolysis and putrefaction. Then it is treated with an acid such as concentrated glacial acetic acid to dissolve the mucus and release the trophoblasts from the mucus. One drawback of using fixative solution and mucus dissolving acid is that the they kill the trophoblast cells and make them not viable for culturing, and preclude prenatal genetic tests that require cell culturing to amplify the genetic materials of individual cells. For example, mosaicism detection needs to determine the genetic makeup of a population of individual fetal cells, the genetic materials of each individual fetal trophoblast cell need to be amplified by culturing to achieve adequate sensitivity. In addition, fixative solution and mucus dissolving acid are causative and tend to damage cell membranes and causes cell contents to spill out. This decreases the number of intact trophoblast cells and precludes genetic testing that requires a large number of intact fetal trophoblasts, such as single point chromosomal mutation detection.

### BRIEF DESCRIPTION OF THE DRAWINGS

Various embodiments of the invention are disclosed in the following detailed description and the accompanying drawings. The drawings are for illustrative purposes and they not necessarily drawn to scale. Not all details or features are included.

FIG. 1 illustrates an example endocervical sample collection device according to various embodiments of the present invention.

FIG. 2 illustrates an example endocervical sample collection device according to various embodiments of the present invention.

FIG. 3 illustrates an example endocervical sample collection device according to various embodiments of the present invention.

FIG. 4 illustrates an example endocervical sample collection device according to various embodiments of the present invention.

FIGS. 5A to 5H are schematic diagrams illustrating cross-sections of different variations of the collection surface according to various embodiments of the present invention.

FIG. 6 shows images of isolated EVT from endocervical sampling.

FIG. 7 shows results of the genome sequencing of isolated EVT.

FIG. 8 shows results of using short tandem repeat (STR) genomic fingerprinting to establish fetal-maternal relationship.

### DETAILED DESCRIPTION

The disclosed invention can be implemented in numerous ways, including as a method; a process; a test; a diagnosis; an apparatus; a system; a device (external and/or implantable); and a composition of matters. A detailed description of one or more embodiments of the invention is provided below along with accompanying figures that illustrate the principles of the invention. The invention is described in connection with such embodiments, but the invention is not limited to any embodiment. In the specification, the various implementations of the invention may be referred to as techniques. In general, the order of the steps of disclosed processes may be altered and one or more steps of disclosed processes may be omitted within the scope of the invention. In addition, if a theory is mentioned or set forth, it is not our intention for it to be limiting or binding to the claimed invention.

We herein disclose improved systems and methods for collecting, isolating and analyzing trophoblasts from the endocervical canal for prenatal genetic testing such as genetic diagnostic testing.

1. Endocervical sample collection device

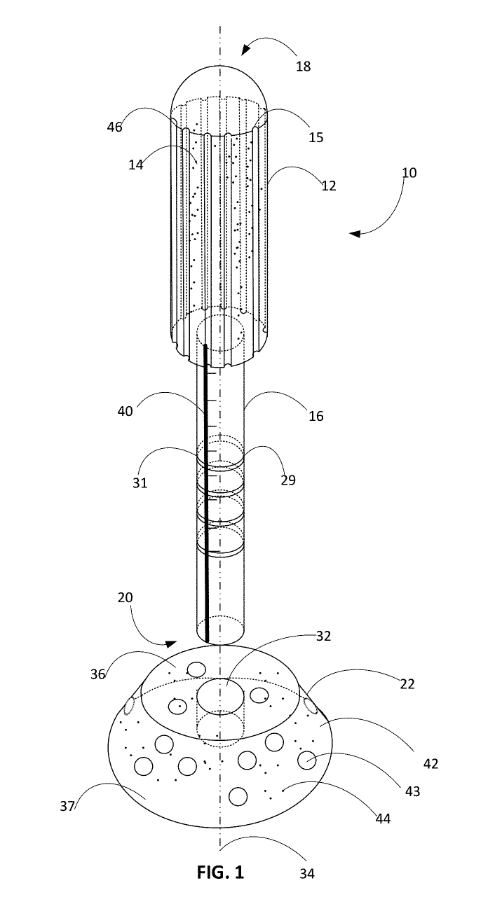

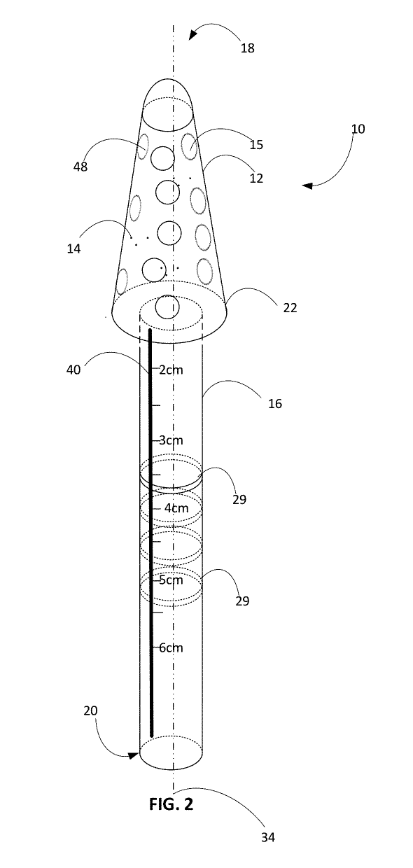

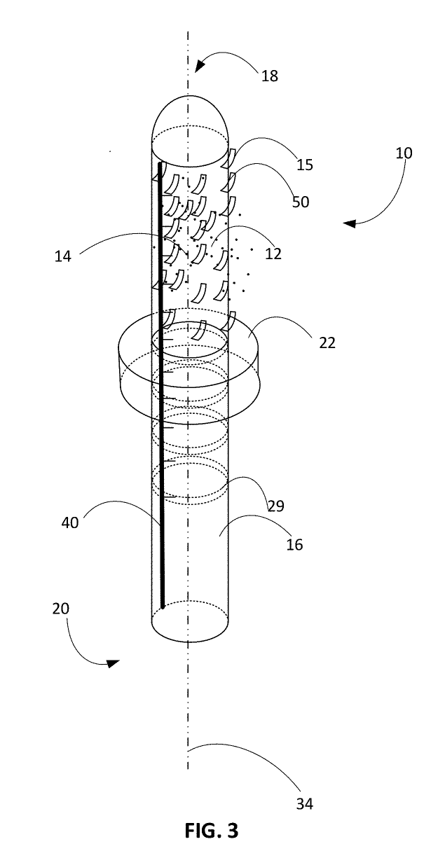

FIGS. 1-4 illustrate different examples of an endocervical sample collection device 10 that includes a collection surface 12 and a handle 16, where the collection surface 12 is located at the distal end 18 of the collection device 10 and the handle is located at the proximal end 20. In various embodiments, the collection device 10 can also include a stopper 22 that is configured to control the distance the collection device 10 can be inserted into the endocervical space. In various embodiments, as shown in FIGS. 1-4, 5A-5H, the collection surface 12 can include one or more layers of polymer coatings 17, surface nanostructures/microstructures 14, chemical and/or biochemical functional groups 11, and/or antibodies 13, which in various embodiments allow the collection surface 12 to preferentially adhere to and pick up fetal trophoblasts as opposed to maternal cells and/or non-cellular materials such as endocervical mucus. In various embodiments, the collection surface 12 can include visually observable macrostructures 15, such as but not limited to, grooves 46 (FIG. 1) or pits or dimples 48 (FIG. 2) soft bristles 50 (FIG. 3) that help to retain and pick up the collected endocervical sample for further isolation and analysis.

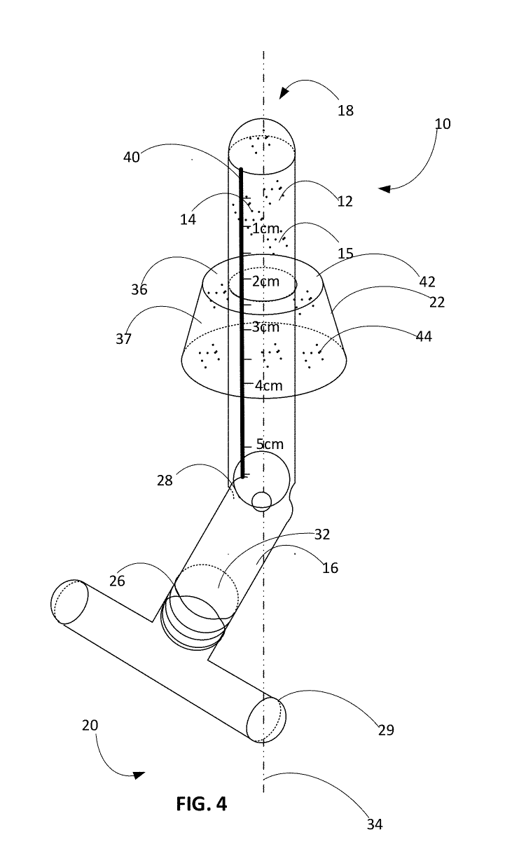

The collection device 10 can assume any suitable shape. In various embodiments, as exemplified in FIG. 2, the collection device 10 includes a conical shaped collection surface 12 attached to an elongated handle 16, where the conical shaped collection surface 12 rounds off at the distal end 18 to minimize the risk of injury to the endocervix during endocervical sample collection process. In various embodiments, as exemplified in FIGS. 1, 3, and 4, the collection device 10 has a cylindrical shaped collection surface 12 connected to an elongated handle 16, where the cylindrical shaped collection surface 12 is rounded off at the distal end 18 to minimize the risk of injury to the endocervix. In various embodiments, as exemplified in FIG. 3, the collection device 10 has a brush-like collection surface 12 with soft bristles 50 such as non-nylon bristles such as silicon bristles connected to an elongated handle 16. The collection surface 12 can be flat (resemble a tongue pressor, not shown) or non-flat, curved or not curved, smooth (FIG. 4) or textured (visually observable) such as grooved (FIG. 1) or dimpled (FIG. 2) or with any other suitable visually observable macrostructures, textures or surface patterns that will help the collection device 10 to retain endocervical sample during the sample collection process.

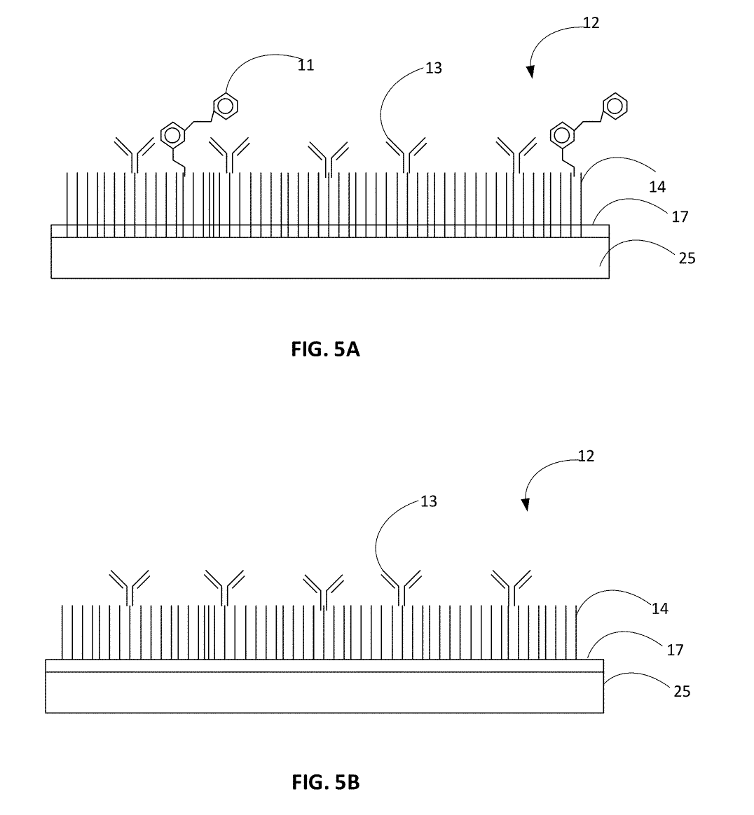

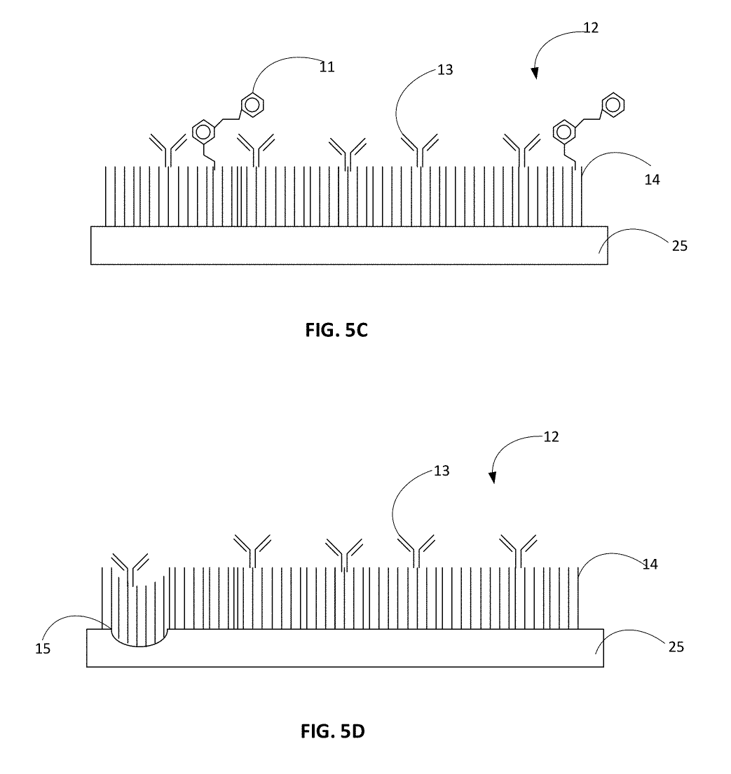

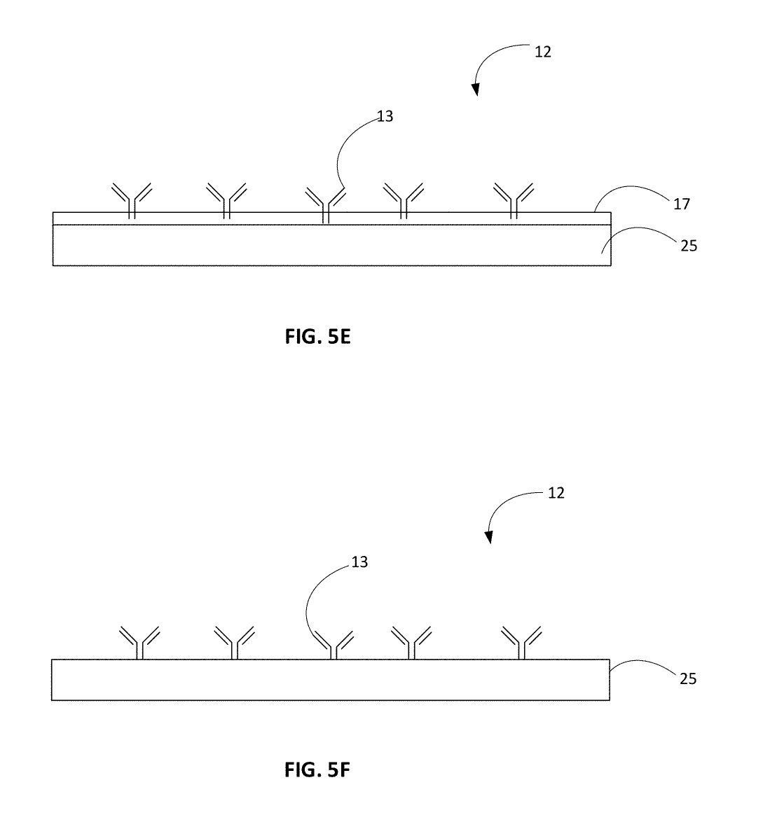

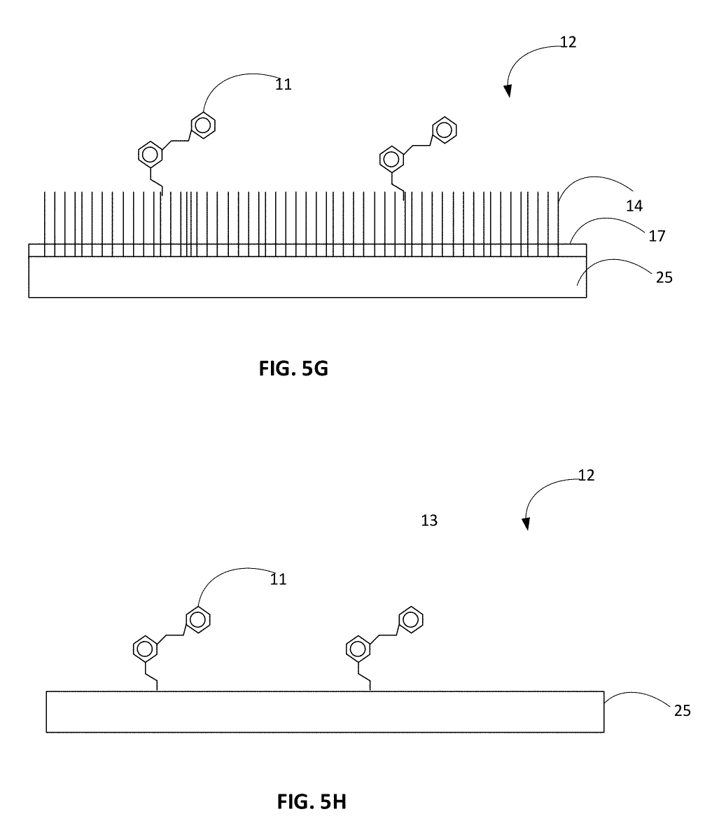

In various embodiments, the polymer coatings 17 on the collection surface 12, as shown in more details in FIGS. 5A, 5B, 5E, 5G, schematic diagrams of a cross-section of the collection surface 12, can serve various functions, such as function to make the collection surface 12 softer and more comfortable to the patient. In various embodiments, the polymer coatings 17 function to allow further modification or functionalization of the collection surface 12, such as a foundation for adding nanostructures and/or microstructures 14, chemical and/or biochemical groups 11, and/or antibodies 13 to the collection surface 12. In various embodiments, the polymer coating 17 is a biocompatible polymer coating that can function to minimize immune response to the collection device 10.

The chemical and/or biochemical groups 11 can be any suitable chemical and/or biochemical groups 11, such as lipids, proteins, sugars and/or DNAs, that allow the collection surface 12 to have improved adhesion to cells, cells with microvilli, and/or fetal trophoblasts, as opposed to non-cellular materials such as endocervical mucus. The improved adhesion to cells allows the collection surface 12 to preferentially or selectively bind to cells, cells with microvilli, and/or fetal trophoblasts, as opposed to non-cellular materials such as endocervical mucus, and in some embodiments, it allows the collection surface 12 to preferentially or preferentially bind to fetal trophoblasts over maternal cells and non-cellular materials such as endocervical mucus.

In various embodiments, the antibodies 13 allow the collection surface 12 to have improved adhesion to cells, cells with microvilli, and/or fetal trophoblasts, as opposed to non-cellular materials such as endocervical mucus, which allows the collection surface 12 to selectively bind to and pick up cells, cells with microvilli, and/or fetal trophoblasts as opposed to non-cellular materials such as endocervical mucus. In various embodiments, the collection surface 12 is coated or modified with antibodies that allow the collection surface 12 to selectively bind to cells with microvilli as opposed to cells without microvilli or non-cellular materials such as endocervical mucus. In various embodiments, the collection surface 12 is coated or modified with antibodies that allow the collection surface 12 to selectively bind to fetal trophoblast cells as opposed to maternal cells or non-cellular materials such as endocervical mucus. In various embodiments, the collection surface 12 can be functionalized with monoclonal antibodies or polyclonal antibodies specific to fetal trophoblasts such as human fetal trophoblasts, for example, by covalently bonding the antibodies to the collection surface 12 or by covalently bonding the antibodies to ligands attached to the collection surface 12. The monoclonal antibodies or polyclonal antibodies can be raised or bought from commercial sources.

In various embodiments, the collection surface 12 is enhanced with surface nanostructures and/or microstructures 14. In various embodiments, the nanostructures and/or microstructures 14 on the collection surface 12 are sized and spaced to selectively or preferentially adhere to and pick up trophoblast as opposed to maternal cells and non-cellular materials from the endocervical canal when the collection device 10 is used to collect samples from the endocervical canal by for example scraping against the endocervical canal wall. We attribute this ability of nanostructures and/or microstructures 14 to preferentially bind to trophoblasts to the interaction between the nanostructures and/or microstructures 14 and the microvilli on the surfaces of fetal trophoblasts. Our experimental observations show trophoblasts adhere more strongly to nanostructures and/or microstructures 14 enhanced collection surfaces 12 with nanoscale or microscale topography than surfaces without nanostructures and/or microstructures 14. In some cases, the adhesion is so strong, after washing with buffer, trophoblasts remained attached to the nanostructures and/or microstructure enhanced surfaces while trophoblasts adhered to surfaces not enhanced with nanostructures and/or microstructures 14 were washed off.

In various embodiments, the nanostructures and/or microstructures 14 on the collection surface 12 are sized and spaced to selectively or preferentially adhere to and pick up cells and/or cells with microvilli, and/or trophoblast as opposed to non-cellular materials from the endocervical canal when the collection device 10 is used to collect samples from the endocervical canal by for example scraping against the endocervical canal wall. We attribute this ability of nanostructures and/or microstructures 14 to preferentially bind to trophoblasts to the interaction between the nanostructures and/or microstructures 14 and the cell surfaces and/or the microvilli on the surfaces of cells.

In various embodiments, the nanostructures and/or microstructure 14 can be grooves, pits, holes, poles, hairs, hooks, protrusions, bumps, bristles, wires, fibers, tubes, particles, protrusions, bristles, meshes, textures, woven or nonwoven fibrous mats, and/or combinations thereof that have nanoscale or microscale cross-sectional diameter or width. In various embodiments, the nanostructures and/or microstructures 14 comprises of nanoscale grooves, pits, holes, poles, hairs, hooks, protrusions, bumps, bristles, wires, fibers, tubes, particles, protrusions, bristles, meshes, textures, woven or nonwoven fibrous mats, and/or combinations thereof that have nanoscale diameter or width. In various embodiments, the nanostructures and/or microstructures 14 comprises of microscale grooves, pits, holes, poles, hairs, hooks, protrusions, bumps, bristles, wires, fibers, tubes, particles, protrusions, bristles, meshes, textures, woven or nonwoven fibrous mats, and/or combinations thereof that have nanoscale diameter or width.

In various embodiments, the nanostructures and/or microstructures 14 can have different average cross-sectional diameters or widths, for example, about 5 nm, about 10 nm, about 25 nm, about 50 nm, about 75 nm, about 100 nm, about 125 nm, about 150 nm, about 200 nm, about 250 nm, about 300 nm, about 350 nm, about 400 nm, about 450 nm, about 500 nm, about 500 nm, about 550 nm, about 600 nm, about 650 nm, about 700 nm, about 750 nm, about 800 nm, about 850 nm, about 900 nm, about 950 nm, about 1 μm, about 1.2 μm, about 1.4 μm, about 1.6 μm, about 1.8 μm, about 2 μm, about 2.5 μm, about 3 μm, about 3.5 μm, about 4 μm, about 4.5 μm, about 5 μm, about 7.5 μm, about 10 μm, about 15 μm, about 20 μm, about 25 μm, about 30 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 120 μm, about 140 μm, about 160 μm, about 180 μm, about 200 μm, about 250 μm, about 300 μm, about 350 μm, about 400 μm, about 450 μm, about 500 μm, about 550 μm, about 650 μm, about 700 μm, about 750 μm, about 800 μm, about 850 μm, about 900 μm, about 950 μm, or about 1000 μm.

In various embodiments, the nanostructures and/or microstructures 14 can have uniform cross-sectional diameter or width. In various embodiments, the nanostructures and/or microstructures 14 can have a range of cross-sectional diameters or widths, for example, from about 5 nm to about 1 μm, from about 5 nm to about 500 nm, from about 5 nm to about 100 nm, from about 5 nm to about 50 nm, from about 5 nm to 20 nm, from about 20 nm to about 250 nm, from about 20 nm to about 200 nm, from about 40 nm to about 200 nm, from about 50 nm to about 150 nm, from about 75 nm to about 100 nm, from about 100 nm to about 500 nm, from about 100 nm to about 1000 nm, from about 1 μm to 10 μm, from about 1 μm to 100 μm, from about 1 μm to about 500 μm, or from about 500 μm to about 1000 μm.

In some embodiments, the nanostructures and/or microstructures 14 can have different average depths, for example, about 0.01 μm, about 0.1 μm, about 0.5 μm, about 1 μm, about 5 μm, about 10 μm, about 25 μm, about 50 μm, about 100 μm, about 125 μm, about 150 μm, about 250 μm, or about 500 μm.

In various embodiments, the nanostructures and/or microstructures 14 can have uniform depths. In various embodiments, the nanostructures and/or microstructures 14 can have a range of depths, for example, from about 0.01 μm to about 0.1 μm, from about 0.1 μm to 1 μm, from about 1 μm to 10 μm, from about 10 μm to 50 μm, from 10 μm to 100 μm, from about 100 μm to about 500 μm, from about 1 μm to about 500 μm, from about 5 μm to about 150 μm, from about 10 μm to about 125 μm, or from about 50 μm to about 100 μm, from about 100 μm to about 500 μm, from about 100 μm to about 1 mm, from about 500 μm to about 2 mm, from about 500 μm to about 3 mm, from about 500 μm to about 4 mm, or from about 500 μm to about 5 mm.

In various embodiments, the nanostructures and/or microstructures 14 can have different average degrees of density, for example, about 0.1 nanostructure and/or microstructure per square μm, about 1 nanostructure and/or microstructure per square μm, 10 nanostructures and/or microstructures per square μm, 25 nanostructures and/or microstructures per square μm, 50 nanostructures and/or microstructures per square μm, 75 nanostructures and/or microstructures per square μm, 100 nanostructures and/or microstructures per square μm, 100 nanostructures and/or microstructures per square μm, 150 nanostructures and/or microstructures per square μm, 200 nanostructures and/or microstructures per square μm, 250 nanostructures and/or microstructures per square μm, 300 nanostructures and/or microstructures per square μm, 400 nanostructures and/or microstructures per square μm, 500 nanostructures and/or microstructures per square μm, 750 nanostructures and/or microstructures per square μm, 1000 nanostructures and/or microstructures per square μm, 1500 nanostructures and/or microstructures per square μm, or 2000 nanostructures and/or microstructures per square μm, about 0.1 nanostructure and/or microstructure per square mm, about 1 nanostructure and/or microstructure per square mm, 10 nanostructures and/or microstructures per square mm, 25 nanostructures and/or microstructures per square mm, 50 nanostructures and/or microstructures per square mm, 75 nanostructures and/or microstructures per square mm, 100 nanostructures and/or microstructures per square mm, 100 nanostructures and/or microstructures per square mm, 150 nanostructures and/or microstructures per square mm, 200 nanostructures and/or microstructures per square mm, 250 nanostructures and/or microstructures per square mm, 300 nanostructures and/or microstructures per square mm, 400 nanostructures and/or microstructures per square μm, 500 nanostructures and/or microstructures per square mm, 750 nanostructures and/or microstructures per square mm, 1000 nanostructures and/or microstructures per square μm, 1500 nanostructures and/or microstructures per square mm, or 2000 nanostructures and/or microstructures per square mm.

In various embodiments, the nanostructures and/or microstructures 14 can have uniform degree of density. In various embodiments, the nanostructures and/or microstructures 14 can have varying degrees of density, for example, from about 0.1 nanostructure and/or microstructure per square μm to about 1000 nanostructures and/or microstructures per square μm, from about 1 nanostructures and/or microstructures per square μm to about 500 nanostructures and/or microstructures 14 per square μm, from about 1 nanostructures and/or microstructures 14 per square μm to about 250 nanostructures and/or microstructures per square μm, from 10 nanostructures and/or microstructures per square μm to 100 nanostructures and/or microstructures per square μm, or from about 50 nanostructures and/or microstructures per square μm to about 100 nanostructures and/or microstructures per square μm, from about 0.1 nanostructure and/or microstructure per square mm to about 1000 nanostructures and/or microstructures per square mm, from about 1 nanostructures and/or microstructures per square mm to about 500 nanostructures and/or microstructures 14 per square mm, from about 1 nanostructures and/or microstructures 14 per square μm to about 250 nanostructures and/or microstructures per square mm, from 10 nanostructures and/or microstructures per square mm to 100 nanostructures and/or microstructures per square mm, or from about 50 nanostructures and/or microstructures per square mm to about 100 nanostructures and/or microstructures per square mm.

In various embodiments, the collection surface 12 is made of, and/or is coated with any suitable material such as silicon, glass, quartz, metal, metal alloys, composite material, inorganic polymers such as polyacrylonitriles (PAN), polyetherketones, polyimides, polyamides and thermoset plastics, organic polymers including protein and/or combination thereof. In various embodiments, the collection surface 12 is made of, and/or is coated with any suitable biocompatible materials such as biocompatible glass, biocompatible polymers, biocompatible metals, biocompatible metal alloys, biocompatible composite materials, and/or combinations thereof.

In various embodiments, the nanostructures and/or microstructures 14 on the collection surface 12 is coated or modified with chemicals or biochemical functional groups 11 such as lipids, proteins, and DNAs that allow the collection surface 12 to have improved adhesion to cells as opposed to non-cellular materials such as endocervical mucus. The improved adhesion to cells allows the collection surface 12 to preferentially or selectively bind to cells as opposed to non-cellular materials such as endocervical mucus. In various embodiments, the cell collection surface is coated or modified with chemicals that allow the collection surface 12 to preferentially or selectively bind to cells with microvilli as opposed to cells with no microvilli and as opposed to non-cellular materials such as endocervical mucus. In various embodiments, the cell collection surface is coated or modified with chemicals or biochemical moieties that allow the collection surface 12 to selectively bind to fetal trophoblast cells as opposed to maternal cells or non-cellular materials such as endocervical mucus.

In various embodiments, the nanostructures and/or microstructures 14 on the collection surface 12 are coated or modified with antibodies 13 hat allow the nanostructures and/or microstructures 14 to have improved adhesion to cells as opposed to non-cellular materials such as endocervical mucus, which allows the nanostructures and/or microstructures 14 to selectively bind to and cells as opposed to non-cellular materials such as endocervical mucus. In various embodiments, the nanostructures and/or microstructures 14 are coated or modified with antibodies 13 that allow the nanostructures and/or microstructures 14 to selectively bind to cells with microvilli as opposed to cells without microvilli or non-cellular materials such as endocervical mucus. In various embodiments, the nanostructures and/or microstructures 14 are coated or modified with antibodies that allow the nanostructures and/or microstructures 14 to selectively bind to fetal trophoblast cells as opposed to maternal cells or non-cellular materials such as endocervical mucus. For example, the nanostructures and/or microstructures 14 can be functionalized with monoclonal antibodies or polyclonal antibodies specific to fetal trophoblasts such as human fetal trophoblasts, for example, by covalently bonding the antibodies to nanostructures and/or microstructures 14. The monoclonal antibodies or polyclonal antibodies can be raised or bought from commercial sources.

In various embodiments, the nanostructures and/or microstructures 14 on the collection surface 12 can be optionally be wholly or partially coated with one or more layers of polymer coatings 17 such as biocompatible polymer coatings. In various embodiments, a layer of polymer coating 17 may cover the bottom half of the nanostructures and/or microstructures 14 and function to protect the nanostructures and/or microstructures 14 during insertion into the endocervical canal, only exposing only the tip of the nanostructures and/or microstructures 14 for attaching to trophoblasts. In one example, the nanostructures and/or microstructures 14 is first embedded (e.g., potted) in a plastic or polymer matrix material such as PTFE, and then the material is partially etched or otherwise partially removed (either in situ or ex situ) such that the plastic or polymer matrix can protect most of the length of each nanofiber, leaving only top portions of the nanostructures and/or microstructures 14 exposed for adhering to trophoblasts.

In various embodiments, the collection surface 12 can be enhanced with various combinations of nanostructures and/or microstructures 14, one or more layers of polymer coatings 17, chemical or biochemical functional groups 11, and/or antibodies 13. FIGS. 5A-5H show some possible combinations, which by no means exhaustive.

Various technology such as nanotechnology or nanomachinery, micro-technology or micro-machinery can be used to fabricate the collection surface 12 and the nanostructures and/or microstructures 14 on the collection surface 12. For example, as shown in FIGS. 5A, 5B, 5C, 5D, 5E, 5F, 5G, 5H, the nanostructures and/or microstructures 14 may be formed by attaching previously formed nanostructures and/or microstructures to an underlying surface or substrate, and/or growing the nanostructures and/or microstructures such as the nanofibers and/or microfibers, or nanowires and/or microwires directly on the collection surface 12. For another example, the nanostructures and/or microstructures 14 on the collection surface 12 are created by controllably depositing and selectively curing curable materials on a planar x-y substrate. The materials are cured through the z-plane of the material. The planar x-y substrate is then used to form the collection surface 12 of the collection device 10 by wrapping it around a substrate forming a body of the of the collection device 10. For another example, the nanostructures and/or microstructures 14 on the collection surface 12 is created by controllably depositing and selectively curing curable materials directly on a non-planar sample collection surface 12 (shown here as a cylindrical surface). In some embodiments, the selective curing technique is through micro photolithography and specific wavelength energy exposure. In various embodiments, the selective curing means is through focused electron beam scanning. The size, spacing, and cross-sectional shapes of the created nanostructures are predetermined based on what is optimal for adhering and picking up fetal trophoblast cells from the endocervical canal.

Other methods for fabricating, coating, and/or functionalizing nanostructures are readily available in various publications, which include but not limited to U.S. Pat. No. 9,725,825, US20140295208, US20140349085, US20140374702, 20050191419, U.S. Pat. No. 8,758,985, 20160161677, U.S. Pat. No. 5,294,465, WO2004092836, U.S. Pat. Nos. 6,709,622, 9,627,199, 8,828,762, 9,221,994, WO201704615, U.S. Pat. Nos. 7,955,486, 8,728,720, 9,001,323, 7,771,790, U.S. Pat. Nos. 9,327,969, 8,557,612, 8,877,542, US20030152759, EP3160898, WO2015031831, WO2014138172, WO2015200496, U.S. Pat. No. 7,628,972, WO2012092015, U.S. Pat. Nos. 7,622,189, 7,485,488, WO2008045114, US20090233236, U.S. Pat. Nos. 7,291,284, 7,846,412, US20110297084, US20070207318, WO2016149711, US20110210480, EP3023386, US20150017433, U.S. Pat. No. 8,815,346, CA2477270, U.S. Pat. No. 9,285,363, US20040018587, US20050265922, WO2013098657, U.S. Pat. No. 6,277,448, EP1802783, US20160200630, U.S. Pat. No. 8,075,682, WO2003018862, US20130216777.

Various well known methods can be used to identify the antibodies used for functionalizing the collection surface 12, the second collection surface 42, the nanostructures and/or microstructures 14 on the collection surface 12, and/or the nanostructures and/or microstructures 14 on the second collection surface 42. In various embodiments, the monoclonal antibodies specific to human fetal trophoblast cells can be identified by screening a large number (e.g., thousands, millions, or billions) of antibodies, either monoclonal or polyclonal, to human fetal trophoblast extracts (e.g., human early placenta extracts) as immunogens. The antibodies that react to the human fetal trophoblasts (e.g. human fetal extra-villous trophoblasts) are identified. The antibodies that are specific to human fetal trophoblasts are identified by excluding antibodies that react to a variety of other human tissues.

There are also various known monoclonal antibodies that specifically react to human fetal trophoblasts. In some embodiments, anti-HLA-G monoclonal antibodies can be used as human fetal trophoblast specific antibodies in healthy individuals, since the non-classical MHC molecule HLA-G is only expressed on extra-villous trophoblast cells not on maternal cells. In some embodiments, FD0161G murine monoclonal antibodies are used as human fetal trophoblast specific antibodies, since FD0161G murine monoclonal antibodies react to extra-villous trophoblast cells in human decidua. In some embodiments, MA-25128 trophoblast protein antibodies are used as human fetal trophoblast specific antibodies, since MA-25128 trophoblast protein antibodies (Thermo Fisher Scientific) reacts with human trophoblast membranes and can detect trophoblast protein in human and non-human samples.

The collection device 10 further includes a handle 16 configured for secure gripping and manipulation of the collection device 10. In various embodiments, the gripping mechanism 29 can be any suitable gripping mechanism, include but not limited to, handle textures, handle groves 31, and/or handle grip bar 24.

In various embodiments, the handle can be adjustable in length and angle to suit individual patient's anatomy and/or or practitioner's preference, the handle 16 can include one or more length adjusting mechanisms 26 (FIG. 4) for adjusting the length of the handle and/or one or more angle adjusting mechanisms 28 (FIG. 4) for adjusting the angle of the handle 16 to the collection surface 12 or the handle angle 30.

In a non-pregnant woman, the cervix is usually 2 to 4 cm long and roughly cylindrical in shape. As pregnancy progresses, especially at late stage, the length of the cervix may change. If the trophoblast collection device enters too far through the cervical internal os, it may risk rupturing the uterus membrane. Therefore, in some embodiments, the collection device 10 can further configured to include a stopping mechanism or stopper 22 configured to limit the maximum distance the collection device can enter the endocervical space. In various embodiments, the stopper 22 is positioned on the handle 16. In various embodiments, the position of the stopper 22 on the handle is adjustable to suit individual patient's anatomy.

In various embodiments, as shown in FIG. 1, the stopper 22 is in the form of a tapered disk with an opening 32, where the stopper 22 is sized and shaped to be excluded from the entrance of the endocervical space from the vagina (known as the external os), where the opening 32 is sized and shaped to allow the stopper 22 to fit snugly over the handle 16, where the position of the stopper 22 along the longitudinal axis 34 of the collection device 10 can be adjusted by moving (e.g., sliding) the stopper 22. Adjusting the position of the stopper 22 along the longitudinal axis 22 controls the maximal distance the collection device 10 can be inserted into the endocervical space during endocervical sample collection. In various embodiments, the diameter of the stopper 22 is smaller on the side of the distal surface 36 facing the distal end 18 (distal to the handle 16), while the diameter of the stopper 22 is larger on the proximal side of the proximal surface 38 facing the proximal end 20 (proximal to the handle 16).

In various embodiments, the stopper 22 is made of, and/or is coated with any suitable material such as silicon, glass, quartz, metal, metal alloys, composite material, inorganic polymers such as polyacrylonitriles (PAN), polyetherketones, polyimides, polyamides and thermoset plastics, organic polymers including protein and/or combination thereof. In various embodiments, the stopper 22 is made of, and/or is coated with any suitable biocompatible materials such as biocompatible glass, biocompatible polymers, biocompatible metals, biocompatible metal alloys, biocompatible composite materials, and/or combinations thereof.

In various embodiments, as shown in FIG. 1, the distal surface 36 and the side surface 37 of the stopper 22 can serve as a second collection surface 42 for collecting endocervical sample, where the second collection surface 42 is configured to have improved adhesion to fetal trophoblast cells similar to the collection surface 12 discussed above. In various embodiments, the second collection surface 42 can include macrostructures 43, nanostructures and/or microstructures 44, chemical and/or biochemical functional groups 52, antibodies 54 on its surface, similar to the collection surface 12. In general, the characteristics, functionalization, and fabrication of the collection surface 12 discussed above can be applied to the second collection surface 42. In general, the characteristics, functionalization, and fabrication of the macrostructures 15 discussed herein can be applied to macrostructures 43 of the second collection surface 42. In general, the characteristics, functionalization, fabrication of the nanostructures and/or microstructures 14 of the collection surface 12 discussed herein can be applied to the nanostructures and/or microstructures 44 of the second collection surface 42. In general, the characteristics, functionalization, and fabrication of the chemical and/or biochemical groups 11 discussed herein can be applied to the chemical and/or biochemical groups 52 on the second collection surface 42. In general, the characteristics, functionalization, and fabrication of the antibodies 13 of the collection surface 12 discussed herein can be applied to the antibodies 54 of the second collection surface 42. FIGS. 5A-5H can be used to describe the second collection surface 42, with the collection surface 12 substituted by the second collection surface 42, the nanostructures and/or microstructures 14 substituted by nanostructures and/or microstructures 44, the chemical and/or biochemical groups 11 substituted by chemical and/or biochemical groups 52, the antibodies 13 substituted by antibodies 54, the macrostructures 17 substituted by macrostructures 44. FIGS. 5A-5H does not represent exhaustive possible combinations of arrangements for the second collection surface.

In various embodiments, as shown in FIGS. 1-4, a visible scale 40 is marked on the collection device 10, for example on the handle 16 of the collection device 10, to indicate how far the collection device 10 can be inserted into the endocervical space. In one example, a scale with marks at 1 cm, 2 cm, 3 cm, 4 cm, 5 cm and 6 cm is printed/marked on the collection device 10, indicating the distance of the tip (the distal end 18) of the collecting device 10 to the stopper 22, and indicating the maximal distance for which the tip of the collecting device can be inserted into the endocervical space before the stopper 22 is stopped at the external os. For example, if the stopper 22 is positioned at the 2 cm mark, the stopper 22 will come up against the endocervical external os from the vagina and prevent the collection device 10 from entering further into the endocervical space when the tip of the collection device is 2 cm into the endocervical space from the external os.

2. Composition for Processing Endocervical Specimen

We disclose a novel mucolytic preparation for treating endocervical samples that will dissolve the endocervical mucus to release individual fetal trophoblasts while keeping the cells such as fetal trophoblasts in the sample intact and/or viable for downstream culturing, processing, or analysis, including genetic analysis. The mucolytic preparation includes a non-caustic mucolytic compound/macromolecule (herein termed non-caustic mucolytic agent). The non-caustic mucolytic agent dissolves the endocervical mucus but is not lethal to the cells. It will keep the cells such as fetal trophoblasts in the sample viable for downstream culturing, processing, or analysis, including genetic analysis. Further, the non-caustic mucolytic agent does not cause as much damages to cell membranes as the traditional mucolytic agents such as glacial acetic acid. It is better at keeping the cell membrane such as trophoblast cell membrane intact for downstream processing and analysis including genetic analysis.

In various embodiments, the non-caustic mucolytic agent dissolves endocervical mucus by depolymerizing mucin glycoproteins. In various embodiments, the mucolytic preparation includes one or a combination of drugs selected from a group of mucolytic drugs including N-acetyl cysteine (NAC), ambroxol, sobrerol, carbocystein, carbocysteine sobrerol, letosteine, cithiolone, iodinated glycerol, N-isobutyrylcysteine, myrtol and erdosteine.

In various embodiments, the mucolytic agent has antioxidant property that can extend protection to the cells such as fetal trophoblasts from being damaged or killed. In various the mucolytic preparation includes an antioxidant that can protect cells such as fetal trophoblasts from being damage or killed.

In various embodiments, the mucolytic preparation includes a mucolytic agent that dissolves mucus presenting in the endocervical sample by depolymerizing DNA and F-actin polymer networks present in mucus. In various embodiments, the mucolytic preparation includes one or more compound selected from the group that includes dornase alfa, gelsolin, and thymosin.

In various embodiments, the mucolytic preparation includes a non-destructive mucolytic agent that dissolves the mucus presenting in the endocervical sample by disrupting the polyionic oligosaccharide mucin network through charge shielding. In various embodiments, the mucolytic preparation includes one or more compounds/macromolecules selected from the group that includes dextran and heparin.

In various embodiments, the non-caustic mucolytic agent is selected from the group consisting of N-acetyl cysteine (NAC), ambroxol, sobrerol, carbocystein, carbocysteine sobrerol, letosteine, cithiolone, iodinated glycerol, N-isobutyrylcysteine, myrtol, erdosteine, dornase alfa, gelsolin, thymosin, dextra, and heparin.

In various embodiments, the mucolytic preparation is a part of a preparation solution for preparing endocervical samples for further processing (e.g., culturing, polymerase chain reaction, sequencing, pathology diagnosis).

In various embodiments, the mucolytic preparation is isotonic and buffered with one or more buffers such as phosphate, Tris, acetate buffer to help to keep the cells intact and/or viable for culturing, further processing and/or testing.

In various embodiments, the mucolytic preparation has antioxidant property and/or includes one or more antioxidants to help to keep the cells intact and/or viable for culturing, processing and/or testing.

In various embodiments, the mucolytic preparation includes one or more chemicals used in a traditional fixative solution such as formaldehyde, methanol, ethanol, acetic acid (e.g., glacial acetic acid), and/or combination thereof to kill off cells/organisms to prevent the endocervical sample treated from degradation, autolysis, and putrefaction.

We further discloses a method of collecting, enriching, and isolating fetal trophoblasts for prenatal genetic testing of a pregnant woman, the method comprises collecting an endocervical sample of the pregnant woman at 5-20 gestational week using an endocervical sample collection device as disclosed in the present invention, where the collection device enriches for fetal trophoblasts during the process of sample collection; isolating the fetal trophoblast cells present in the endocervical sample; and conducting genetic testing of the isolated trophoblast cells. In various embodiments, the surface of the endocervical sample collection device is enhanced with nanostructures and/or microstructures as described herein in various embodiments. In various embodiments, the surface of the endocervical sample collection device is further functionalized with fetal trophoblast specific antibodies as described herein in various embodiments. In various embodiments, where isolating the fetal trophoblast cells present in the endocervical sample comprises treating the endocervical sample with a non-caustic mucolytic preparation as disclosed herein in the present invention to release intact and viable fetal trophoblast cells; isolating individual viable fetal trophoblast cells using laser capture microdissection; and culturing the individual fetal trophoblast cells to increase the genetic materials of the individual trophoblast cells. In various embodiments, the non-caustic mucolytic agent is selected from the group consisting of N-acetyl cysteine (NAC), ambroxol, sobrerol, carbocystein, carbocysteine sobrerol, letosteine, cithiolone, iodinated glycerol, N-isobutyrylcysteine, myrtol, erdosteine, dornase alfa, gelsolin, thymosin, dextra, and heparin. In various embodiments, isolating the fetal trophoblast cells present in the endocervical sample comprises isolating individual fetal trophoblast cells present in the collected endocervical sample by running the endocervical sample through a polymer microchip such as nanoVelcro chip, an example of which is described in the present invention. In various embodiments, the method further comprising conducting fetal genetic screening for various genetic abnormalities based on the genetic testing result of the isolated fetal trophoblast cells. In various embodiments, the method further comprising predicting risk for genetic birth defects based the genetic testing result of the isolated fetal trophoblast cells. In various embodiments, the method further comprising predicting pregnancy risk based on the genetic testing result of the isolated fetal trophoblast cells. In various embodiments, the method further comprises conducting fetal genetic diagnosis based on the genetic testing result of the isolated fetal trophoblast cells.

### Example 1. Example Design of a Endocervical Sample Collection Device

FIG. 4 illustrates an example endocervical sample collection device 10 that is in the form of a cylindrical shaped rod. The tip (distal end 18) of the collection surface 12 is rounded off to prevent potential injury to uterus lining and endocervix. The collection device 10 includes collection surface 12, a handle 16 and a stopper 22 in the form of a sliding plug. In this example, the collection surface 12 is made of glass etched with nanostructures and/or microstructures 14 with nanoscale and/or microscale topography and then functionalized with human fetal trophoblast specific anti-HLA-G monoclonal antibodies 13. The collection device 10 is conducive for preferentially adhering to and picking up human fetal trophoblast, as opposed to non-trophoblast cells and non-cellular materials, when the collection device 10 is rotated to gently scrape against the walls of the endocervix to collect endocervical samples.

The handle 16 is attached to the proximal end of the collection surface 10. In this example, the handle 16 is an extension of the collection surface 12, it is made of the same glass material as the collection surface 12. But unlike the collection surface 12, it not etched with nanostructures. Rotating the handle 16 allows the collection surface to be rotated to collect endocervical sample from the endocervix. The handle 16 is rippled to provide a better grip.

In this example, the stopper 22 is tapered with a circular hole in the center. The circular hole is shaped and sized in such a way that allows the stopper 22 to snugly fit over the handle 16 or the collection surface 12. The stopper 22 includes a circular distal surface 36 and a side surface 37. The diameter of the distal surface 36 is smaller than the diameter of the proximal surface 42, reflecting the tapered shape of the stopper 22.

The position of the stopper 22 can be adjusted along the length of the handle 16 and/or the collection surface 12 to change the maximum distance the collection device 10 will be allowed to enter the endocervix. A visible scale 40 is marked on the collection device (on the collection surface and on the handle) to indicate how far the collection device can travel up the endocervix when the stopper 22 is placed at that position. In this example, 1 cm, 2 cm, 3 cm, 4 cm, 5 cm and 6 cm marks are printed/marked on the collection device 10, indicating the distance of the tip of the collecting device 10 to the stopper 22. The mark also indicates the maximum distance the tip of the collection device 10 can travel into the endocervix before the stopper 22 is stopped at the external os of the endocervix when the stopper 22 is positioned at that mark position. For example, if the sliding plug is positioned at the 2 cm mark (as shown here), the sliding plug will come up against the external os of endocervix from the vagina and prevent the collection device 10 from entering further into the endocervix when the tip of the collection device 10 is 2 cm into the endocervix from the vagina.

The collection device 10 also includes a stopping mechanism that allows the stopper to remain in the position it is positioned without being dislodged when slight force is applied. This ensures that the stopper 22 will remain in its position during sample collection. In this example, the stopper 22 is made of a pliable rubber, the diameter of the circular hole is slightly smaller than the diameter of the handle to ensure a snug fit. Pressure and fiction generated when the stopper 22 is fitted over the handle 16 serve as the stopping mechanism to keep the stopper in its position once positioned.

The stopper 16 may include a second collection surface 42, which includes the distal surface 36 and the side surface 37. The second collection surface 42 is configured to collect endocervical samples containing fetal trophoblasts from the space just outside of the endocervix (e.g., at the level of external os) when the collection device 10 is inserted into the cervix and rotated to collect endocervical sample. The second collection surface 42 includes a glass surface and is etched with nanostructures and/or microstructures 44 and functionalized with human fetal trophoblast specific anti-HLA-G monoclonal antibodies 13 (not shown). The second collection surface 42 preferentially adhere to human fetal trophoblast cells as opposed to non-trophoblast cells and non-cellular materials.

The length of the handle is adjustable using a handle length adjusting mechanism 26 to suit the anatomy of the patient. The neck of handle 16 is flexible or adjustable in angle using an angle adjusting mechanism 28. If the internal os of endocervix is facing the patient's anterior vaginal wall (anteverted) or posterior vaginal wall (retroverted) instead of facing the vaginal opening, the angel between collection tip and the rest of the handle may be adjusted instead of 180 degree so that the collection tip may be thread into the endocervix.

The handle further includes a handle gripping mechanism 29 in the form of a cross-bar for secure gripping of the collection device 10.

### Example 2. A Method of Fabricating Nanostructures Using a Lithographic Free Method

In this example, a conical shaped soda-lime-silica glass rod with rounded off tip is first prepared with conventional glass technology. Nanostructures on the glass surface are then created using a lithographic-free method.

The glass surfaces are first cleaned with acetone, methanol and 2-propanol. The surfaces of the glass rod are then coated with 1-μm-thick sacrificial layer of SiO₂ using plasma-enhanced chemical vapor deposition (PECVD) with a mixture of N₂O gas and SiH₄ gas. After coating, the glass surfaces are then etched with CF₄ plasma for 5 to 60 min (adjusted for the optimum result) by PECVD. The gas pressure and bias voltage are maintained at 30 mTorr and −600Vb, respectively. After CF₄ plasma etching, nanostructures are developed on the SiO₂ layer-coated glass via the well-known method of preferential etching with a reactive ion. During etching, the sacrificial layer of SiO₂ first evolved into pillar shaped with a high aspect-ratio, 6.6:1, and with subsequent continuous CF₄ etching, the SiO₂ nanopillars serve as etching masks for the selective etching of the glass surface. The sacrificial SiO₂ layer is ultimately etched away, providing a glass surface with hierarchical nanostructures with 15-to-30 nm wide pillars. The hydrophilicity or of the surface can be increased by depositing varying amount of HMDSO (C:H:Si:O) using PECVD. The gas pressure is fixed at 10 mTorr, the bias voltage was −400 Vb, and the deposition time can be varied based on the amount of HMDSO to be deposited (3 to 20 sec).

### Example 3. A Method of Fabricating Carbon Nanotubes

Micropatterned vertically aligned carbon nanotubes can be grown on a silicon substrate with SiO₂. Photolithography is used to deposit silicon substrate with SiO₂. Photolithography is used to deposit catalyst in square patterns of 50-500 μm edge on a SiO₂ wafer. The catalyst layer consists of a 10 nm thick aluminum (Al), which acts as a buffer layer, and 1.5 mm thick iron catalyst layer, which forms nanosize particles for catalytic growth of carbon nanotubes. Ethylene (flow rate of 50-150 standard cc min) is used as a carbon source, and the reaction is carried out at 750° C. An Ar/H₂ gas mixture (15% H₂) with a flow rate of 1300 standard cc per min is used as the buffer gas. Water vapor with a dew point of −20° C. is introduced in the reaction furnace by Ar/H₂ flow during carbon nanotube growth. The growth time is 3 min, and the length of the carbon nanotubes is 100 μm in height. These samples have the optimum dimensions needed for both adhesion and self-cleaning. The hydrophilicity of the surface can be increased by depositing varying amount of HMDSO (C:H:Si:O) using PECVD. The gas pressure was fixed at 10 mTorr, the bias voltage was −400 Vb, and the deposition time can be varied based on the amount of HMDSO to be deposited (3 to 20 sec).

Micropatterned vertically aligned and curved carbon nanotubes can be grown on a silicon substrate with SiO₂ using similar method. Helical or coiled carbon nanotubes are formed by introducing pairs of five-membered and seven-membered rings into the basic six-membered rings contained in the nanotubes. Since the pH values have a definite influence on the yield of coiled carbon nanotubes, a basic (e.g. 8-9) value may be maintained in all preparations by for example using aqueous ammonia vapor with a dew point of −20° C. is introduced in the reaction furnace by Ar/H₂ flow during carbon nanotube growth. The hydrophilicity of the surface can be increased by depositing varying amount of HMDSO (C:H:Si:O) using PECVD. The gas pressure was fixed at 10 mTorr, the bias voltage was −400 Vb, and the deposition time can be varied based on the amount of HMDSO to be deposited (3 to 20 sec).

### Example 4. A Method of Attaching Anti-HLA-G Antibodies to Nanostructure Enhanced Surface

The nanostructure enhanced glass endocervical sample collection surface is first washed well using buffer (PBS) to remove dirt and debris. A nanostructure enhanced glass endocervical sample collection surface is incubated with anti-human HLA-G antibodies at a concentration of 200 mcg/mg in 0.1M phosphate buffer, pH 7.4 on in a shaking bath at 22° C. for 18 hours. Shorter incubation duration may be adequate, but we selected the longer incubation duration to ensure maximum antibody conjugation. Unbound HLA-G-antibodies are then removed by washing with 0.1M phosphate buffer.

### Example 5. A Method of Collecting and Preparing Endocervical Samples to Obtain Viable Trophoblast Cells

A endocervical sample collection device enhanced with nanostructures and functionalized with anti-human HLA-G antibodies is used to scrap against the endocervical wall of pregnant patient (e.g., at 5-20-week gestation) to collect the endocervical specimen.

The collection surface of the collection device is then immersed in a vial containing 25 ml of the disclosed mucolytic composition, which in this example includes an isotonic Ca⁺⁺ and Mg⁺⁺ free PBS Buffer containing 0.1 mg/ml mucolytic drug NAC.

The vial is then agitated using a shaker for 5-30 minutes in a 37° C. incubator. Mucus is dissolved in the process and the freed trophoblast cells are attached to the cell collection surface of the nanostructure enhanced trophoblast collection device. The collection surface has nanoscale topography and conjugated anti-HLA-G antibodies, and preferentially bind to human fetal trophoblast cells as opposed to maternal cells and non-cellular materials.

The collection surface of the endocervical sample collection device is then washed with Ca⁺⁺ and Mg⁺⁺ free PBS Buffer three times. Since the collection surface preferentially binds to human fetal trophoblast cells, the suspended viable cells are enriched in fetal trophoblast cells (trophoblast cell enriched cells).

The trophoblasts attached to the collection surface can be identified using immunofluorescence microscopy, isolated using laser capture microdissection (LCM), and/or released from the cell collection surface using digestive enzymes such as trypsin.

### Example 6. Releasing Trophoblast from Cell Collection Surface Using a Preparation Containing Non-Caustic Mucolytic Compound Trypsin

To release the trophoblast cells attached to the collection surface of the disclosed nanostructure enhanced collection device, collection surface is first washed well with a Ca⁺⁺ and Mg⁺⁺ free PBS buffer. The collection device is then covered in Ca⁺⁺ and Mg⁺⁺ free PBS containing 0.025% trypsin-EDTA (adjusted to pH 7.4 to 7.6), placed in 37° C. incubator for 1-2 minutes. The trypsin coated cells attached to the collection surface are allowed to detach during incubation. Serum or medium containing serum is added to the cell suspension as soon as cells become detached to inhibit further tryptic activity which may damage cells. Detached cells (viable) are then suspended by gently pipetting the cell suspension to break up the clumps. The suspended cells can then be further processed: purified, cultured (if viable), counted, isolated, identified, sequenced, colored, examined, fixed and/or combinations thereof.

### Example 7. Isolating Individual Trophoblast Cells Using Laser Capture Microdissection

Trophoblast enriched cell population are fixed using fixative solution (if this has not been done) and spread on a glass slide covered with a thin layer of agar that does not interfere with laser sectioning. The slide is labeled overnight at 4° C. with 10 μg/mL of primary antibody that selectively bind to trophoblast cells (e.g., extra-villous trophoblast cells). The primary antibody is labeled and visualized using fluorescein isothiocynate (FITC) conjugated secondary antibodies. Trophoblast cells are identified using immunofluorescent microscopy and isolated using a commercially available laser capture microdissection system such as ArcturusXT™ LCM System (Thermo Fisher Scientific). A plastic cap is placed atop the slide section containing trophoblast cell of interest, a laser beam is focused and fired at the bottom of the cap melting plastic directly onto the cell region of interest. The cap is then removed, and with it the trophoblast cell of interest. Trophoblast cells isolated using laser capture microdissection are collected and pooled in a collection tube. The isolated trophoblast cells can then be subjected to various downstream microgenomics procedures such as next-generation sequencing, Sanger sequencing, PCR, and proteomics.

### Example 8. A Method of Screening for Fetal Chromosomal Aneuploidy

Most cells in human body have 23 pairs of chromosomes or a total of 46 chromosomes. (The sperm and egg, or gametes, each have 23 unpaired chromosomes, and red blood cells have no nucleus and no chromosomes.) Aneuploidy is the presence of an abnormal number of chromosomes in a cell, for example for a human cell that has 45 or 47 chromosomes instead of the usual 46. An extra or missing chromosome is a common cause of genetic disorders. Aneuploidy originates during cell division when the chromosomes do not separate properly between two cells. Chromosome abnormalities are detected in 1 of 160 live human births. Apart from sex chromosome disorders, most cases of aneuploidy result in death of the developing fetus (miscarriage). The most frequent aneuploidy in humans is trisomy 16, although fetuses affected with the full version of this chromosome abnormality do not survive to term, some do survive (it is possible these surviving individuals to have the mosaic form, where trisomy 16 exists in some cells but not all). The most common aneuploidy that infants can survive with is trisomy 21, 18 and 13. Trisomy 21, which is found in Down syndrome, affecting 1 in 800 births. Trisomy 18, which is found in Edwards syndrome, affecting 1 in 6000 births, and trisomy 13, which is found in Patau syndrome, affecting 1 in 10,000 births. 10% of infants with trisomy 18 or 13 reach 1 year of age. The American College of Obstetricians and Gynecologists (ACOG) has started to recommend that fetal chromosomal aneuploidy screening be offered to all women regardless of age or risk status.

### Example 9. Screening for Mosaicism in Chromosomal Aneuploidy

Chromosomal mosaicism occurs when changes in chromosome number occurs in some fetal cells and does not occur in all fetal cells. Individuals with mosaicism for a chromosomal aneuploidy tend to have a less severe form of the syndrome resulting from missing or extra chromosome. Screening for chromosomal aneuploidy mosaicism in fetus will provide an important piece of information for would-be parents.

### Example 10. Fetal Genome Wide Sequencing for Detecting Fetal Single Nucleotide Mutation

According to the World Health Organization, single-gene disorders, such as thalassemia and sickle cell anemia are a major health burden. Demographic shifts including advanced maternal age (>35 years), increase the overall risk for fetal chromosomal abnormalities that cause congenital birth defects. Currently, we lack simple, reliable, and safe approaches to screen genetic disorder at single-nucleotide resolution early. Prenatal genetic testing based on cell-free fetal DNA in maternal blood is limited in its reliability for probing a broad range of major genetic anomalies due to the small fetal fraction (4 to 10% at 10 weeks of gestation) and fragmentation of the degrading DNA (146 base pair). Digital polymerase chain reaction (PCR) assays can detect single-gene disorders such as sickle cell anemia and x-linked hemophilia but rely on exact quantification of the fetal fraction, which can be problematic in the first trimester because of the overwhelming amount of co-purifying maternal DNA. To detect gene mutation at single-nucleotide resolution using next generation sequencing, sufficient pure and intact fetal cells are needed. The system and method disclosed herein can capture fetal trophoblast cells in numbers sufficient for such purpose as early as 5 weeks of gestation and therefore when coupled with next generation sequencing may provide a good approach for detecting single-gene disorders early in the pregnancy.

DNA can be extracted from the isolated pure trophoblasts obtained using the systems and methods disclosed herein. Targeted sequencing using next generation sequencing technology are performed after digestion of exogenous DNA. Fetal trophoblast cells are suspended in PBS, deposited on microscope slides, and dried on a slide warmer at 40° C. Before DNA extraction, the slides were immersed in pepsin solution (0.01 g in 100 ml of 0.1N HCl) for 11 min, followed by a PBS wash for 5 min to remove cell membranes and contaminating maternal DNA fragments. Exogenous DNA was further eliminated from the glass-bound nuclei by adding 10 μl of washed, immobilized DNase (DNase I, immobilized on Matrix F7M, MoBiTec) onto the pepsin-treated slides and incubating for 5 min at room temperature. The slides were washed with PBS to remove the beads and terminate DNA digestion. The nuclei were lysed by overnight incubation at 65° with 0.5 μl of Arcturus PicoPure DNA lysis buffer (Applied Biosystems) and then inactivated at 95° C. for 30 min.

For DNA sequencing, Illumina's Nextera tagmentation technology (Nextera DNA Sample Prep Kit, Illumina, San Diego, Calif.) in which a transposase enzyme simultaneously fragments and inserts adapter sequences into dsDNA is used to prepare DNA library. The length of the library insert can be adjusted by increasing or decreasing the time of the digestion reaction. A pool size of around 200 bp is selected and sequenced on a MiSeq FGx system (Illumina). Common SNP loci are analyzed using ForenSeq with primer mix A (e.g., 94 SNV loci and 59 STR loci on all human chromosomes).

For mRNA sequencing, SMARTer Ultra Low RNA Kit (Clontech, Moutain View, Calif.) is used to prepare RNA-seq library. A full-length cDNA is synthesized from RNA with a fixed 3′ and 5′ sequenced added so that the entire cDNA library (average 2 kb in length) can be ampliefied in long distance PCR (LD-PCR). This amplified double-stranded cDNA is then fragmented by acoustic shearing to the appropriate size and used in a standard Illumina library preparation (involving end-repair and kination, A-tailing and adapter ligation, followed by additional amplification by PCR).

### Example 11. Screening for Mitochondria Mutation

Mitochondrial disease is a group of disorders caused by dysfunctional mitochondria, the organelles that generate energy for the cell. Mitochondria are found in every cell of the human body except red blood cells, and convert the energy of food molecules into the ATP that powers most cell functions. Mitochondrial diseases are sometimes (about 15% of the time) caused by mutations in the mitochondrial DNA that affect mitochondrial function. Other causes of mitochondrial disease are mutations in genes of the nuclear DNA, whose gene products are imported into the mitochondria (mitochondrial proteins) as well as acquired mitochondrial conditions.

Although research is ongoing, treatment options are currently limited; vitamins are frequently prescribed, though the evidence for their effectiveness is limited. Membrane penetrating antioxidants, such as the mitochondria-targeted antioxidant MitoQ (mitoquinol mesylate) have the most important role in improving mitochondrial dysfunction. Pyruvate has been proposed recently as a treatment option. N acetylcysteine reverses many models of mitochondrial dysfunction. No definite cure is available. Many of the patients die at young childhood.

Early detection is important for prevention from delivery. The trophoblast collect by our device can be applied to detect the DNA mutation in chromosome and mitochondria DNA after sequencing.

### Example 12. Screening for Cervical Cancer and Pre-Cancer Cell, Human Papilloma Virus (HPV), and Chlamydia and Gonorrhea Infection

Cervical cancer is one of the most common female cancer. It develops from precancer after many years of persistent high risk type of HPV infection. Pap smear is a routine Gynecological exam by checking the morphology and HPV existence of the squamous cell from the cervix surface. At the same time, chlamydia and gonorrhea, which are the two common sexually transmitted disease can be detected from the cell collected from endocervix. The sample collected from the tip and plug can be used for Pap smear, HPV, chlamydia, and gonorrhea screening.

### Example 13. Further Enrichment, Isolation, and Analysis of the Collected Endocervical Samples Using NanoVelcro Microchip

Sample Preparation

The collected endocervical samples were transferred at room temperature to the laboratory, where they were acidified with 500 μl of glacial acetic acid for 5 minutes to dissolve the mucous. Samples were centrifuged at 400×g for 5 minutes at 4° C. The supernatant was removed and the cell pellet was re-suspended in 20 ml of ice-cold sterile phosphate buffered saline (PBS). Specimens were then washed by centrifugation at 190 g and re-suspension three times with 20 mL of PBS, and on the final wash, the specimen was brought to 10 mL with PBS at 4° C.

Isolation of Extravillous Trophoblast (EVT)

The prepared endocervical sampling in 200 μL was injected into the imprinted poly(lactic-co-glycolic acid (PLGA) nanoVelcro microchip at a flow rate of 1.0 mL h-1. After the suspension sample solution containing EVTs was fully flowed through the imprinted PLGA nanoVelcro microchip, the on-chip enriched EVT were first fixed with ethanol (95%) at a flow rate of 1.0 mL h-1 for 10 min. The imprinted PLGA nanoVelcro microchip was briefly washed with washing buffer for 10 min at a flow rate of 1.0 mL h-1. Then, the captured EVT were identified by a three-color immunohistochemistry (ICC) protocol for parallel staining of DPI, anti-CK7 (PE), anti-HLA-G (FITC) to distinguish EVT from nonspecifically captured cervical cells on the nanoVelcro substrates.

Isolation of Single EVT by Laser Capture Microdissection (LCM)

Before the Laser Capture Microdissection (LCM) of single EVT, the target EVTs (DAPI+/CK7+/HLA-G+) captured by the imprinted PLGA NanoVelcro microchip were first identified and registered using the first fluorescent microscope (Nikon Ni) in conjunction with an auto-scan imaging software (Nikon, Element). Then a second microscope, the ArcturusXT™ LCM System (Applied Biosystems™) was utilized to selectively dissect identified EVTs. Afterwards, a CapSure™ HS Cap was placed on top of the region of identified EVTs. Then, an 810 nm IR laser beam was applied to melt the polymer membrane on the cap. The resultant conical polymer pillar, so called sticky finger, dropped down and adhered onto the imprinted PLGA NanoVelcro substrate. In the following, a 355 nm UV laser beam was utilized to cut through the imprinted PLGA NanoVelcro substrate in a designed route around EVT excluding surrounding cervical cells (DAPF/CK-7+/HLA-G−). Finally, the dissected EVTs was removed from the HS Caps and kept in 4-μL PBSsc (REPLI-g Single Cell Whole Genome Amplification Kit, QIAGEN) in a 0.5 mL PCR tube at −20° C. until next step whole genome amplification (WGA) is performed.

Whole Genome Amplification (WGA) of cTBs, Followed by Microarray Analysis

The single EVTs isolated by Laser Capture Microdissection (LCM) was then subjected to subsequent gDNA extraction and amplification using the REPLI-g Single Cell Whole Genome Amplification Kit (QIAGEN, Valencia, Calif.) according to the recommended standard protocol. After further purification (QIAquick PCR Purification Kit, QIAGEN, Valencia, Calif.). 1.0 μg of WGA DNA product was applied for microarray analysis (Agilent, SurePrint G3 Human Catalog 8×60K CGH microarrays).

Short Tandem Repeat (STR) Genomic Fingerprinting

Finally, we utilized short tandem repeat (STR) genomic fingerprinting to establish fetal-maternal relationship between the individually isolated EVT and the matching maternal cervical cells. Amplification of STR polymorphisms in both EVTs gDNA samples and maternal gDNA samples (from cervical cells DAPI+/CK-7+/HLA-G−) were performed by direct PCR method, which was conducted by using GenePrint® 10 System (Promega, Madison, Wis., USA) according to the standard protocol. The GenePrint® 10 System allows co-amplification and three-color detection of ten human loci, including TH01, TPDX, vWA, CSF1PO, D16S539, D7S820, D13S317, D5S818, D21S11 as well as Amelogenin. For STR genotyping, the PCR products were subjected to allele analysis at each locus in Genoseq and identified using Peak Scanner™ Software 2.0. All samples were tested in duplicate.

Results

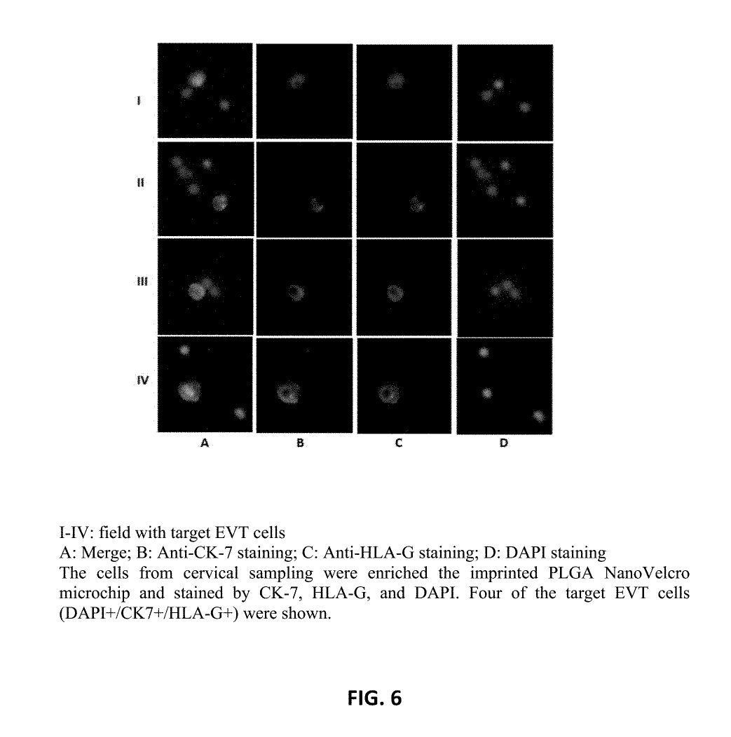

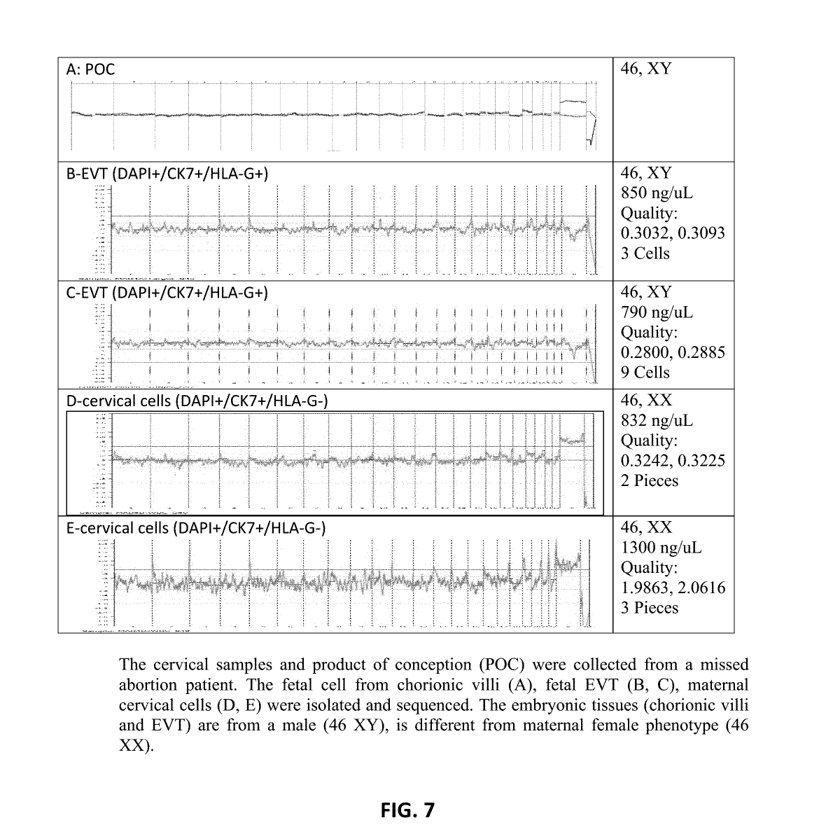

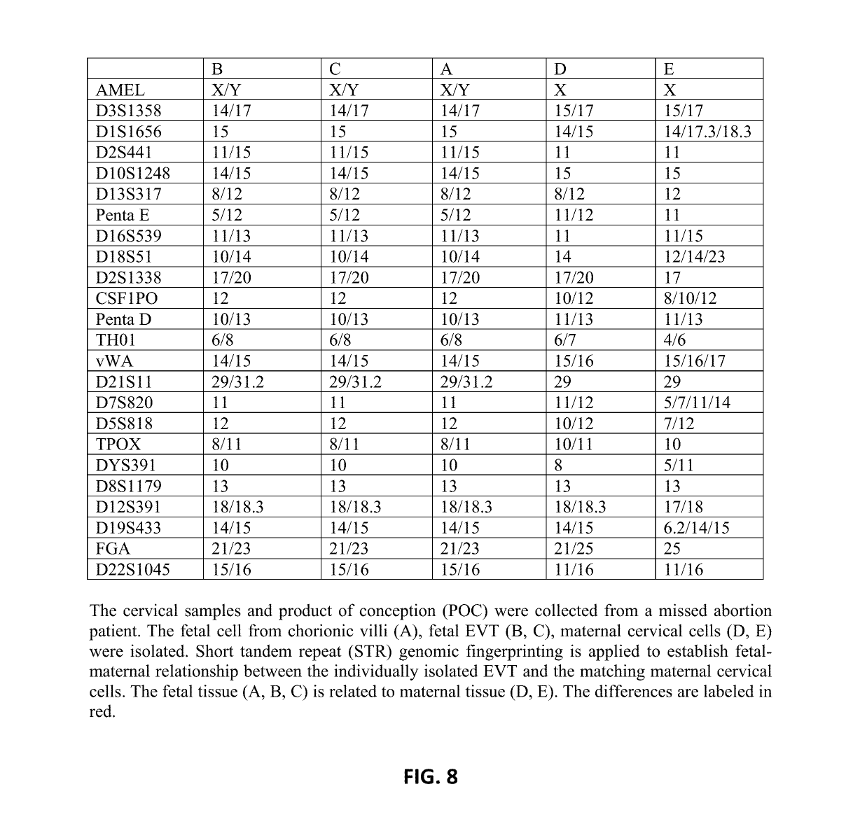

FIG. 6 shows images of isolated EVT from endocervical sampling. FIG. 7 shows results of the genome sequencing of isolated EVT. FIG. 8 shows results of using short tandem repeat (STR) genomic fingerprinting to establish fetal-maternal relationship.

It is intended that the specification and examples be considered as exemplary only, with a true scope and spirit of the invention being indicated by the following claims. In addition, where this application has listed the steps of a method or procedure in a specific order, it may be possible, in certain circumstances, to change the order in which some steps are performed, and it is intended that the particular steps of the method or procedure claims set forth here below not be construed as being order-specific unless such order specificity is expressly stated in the claim.

The subject matter of the present disclosure includes all novel and nonobvious combinations and sub-combinations of the various processes, systems, devices, composition of matters, and configurations, and other features, functions, acts and/or properties disclosed herein, as well as any and all equivalents thereof.

The disclosed embodiments and various details are merely illustrative and are not to be construed as limiting and the invention can be implemented in various alternative ways. Further, the scope of the invention is limited only by the claims and the invention encompasses numerous alternatives, modifications and equivalents.

What is claimed:

1. An endocervical sample collection device for collecting samples from endocervical canal, comprising:
a endocervical sample collection surface configured to scrape samples from the endocervical canal when the collection device is inserted into the endocervical canal, where the collection surface is coated with nanostructures and/or microstructures.

2. The device of claim 1, wherein the nanostructures and/or microstructures comprise of nanostructures having nanoscale diameters or widths.

3. The device of claim 2, the nanostructures and/or microstructures comprise of nanoscale structures selected from the group consisting of nano-grooves, nano-pits, nano-holes, nano-poles, nano-hairs, nano-hooks, nano-protrusions, nano-bumps, nano-bristles, nano-wires, nano-fibers, nano-tubes, nano-particles, nano-meshes, nano-textures, nano-woven or nano-nonwoven fibrous mats, and/or combinations thereof that have nanoscale diameters or widths.

4. The device of claim 2, wherein the nanostructures and/or microstructures are selected from the group consisting of nanoscale nanowires, nanotubes and nanopits

5. The device of claim 1, wherein the nanostructures and/or microstructures comprise of microstructures having microscale diameters or widths.

6. The device of claim 5, the nanostructures and/or microstructures comprises of microscale structures selected from the group consisting of micro-grooves, micro-pits, micro-holes, micro-poles, micro-hairs, micro-hooks, micro-protrusions, micro-bumps, micro-bristles, micro-wires, micro-fibers, micro-tubes, micro-particles, micro-meshes, micro-textures, micro-woven or nonwoven fibrous mats, and/or combinations thereof that have microscale diameter or width.

7. The device of claim 6, wherein the nanostructures and/or microstructures are selected from the group consisting of microscale micro-wires, micro-tubes and micro-pits.

8. The device of claim 1, wherein the nanostructures and/or microstructures are further functionalized with trophoblast specific antibodies.

9. The device of claim 1, wherein the nanostructures and/or microstructures are further functionalized with antibodies specific to human fetal extra-villous trophoblasts.

10. The device of claim 9, wherein the nanostructures and/or microstructures are further functionalized with anti-HLA-G monoclonal antibodies.

11. The device of claim 9, wherein the nanostructures and/or microstructures are further functionalized with FD0161G murine monoclonal antibodies.

12. The device of claim 9, wherein the nanostructures and/or microstructures are further functionalized with MA-25128 trophoblast protein antibodies.

13. The device of claim 1, wherein the device includes a stopping mechanism that sets the maximum distance the tip of the collection device can be inserted into the endocervix from the vagina.

14. The device of claim 1, wherein the device further includes a scale with distance marks, where a distance mark indicates the maximum distance the tip of the collection device can be inserted into the endocervix from the vagina when a stopping mechanism is set at the distance mark.

15. The device of claim 1, wherein the device further includes a handle for providing secure gripping of the collection device during operation, a handle length adjuster for adjusting the length of the handle, and a handle angle adjuster for adjusting the angle of the handle.

16. A preparation for treating sample collected from endocervical canal, where the preparation is not lethal to cells and will keep cell membrane intact.

17. The preparation of claim 16, wherein the preparation includes a non-caustic mucolytic agent selected from the group consisting of N-acetyl cysteine (NAC), ambroxol, sobrerol, carbocystein, carbocysteine sobrerol, letosteine, cithiolone, iodinated glycerol, N-isobutyrylcysteine, myrtol, erdosteine, dornase alfa, gelsolin, thymosin, dextra, and heparin.

18. A method of collecting, enriching, and isolating fetal trophoblasts for prenatal genetic testing of a pregnant woman, comprising:
collecting an endocervical sample of the pregnant woman at 5-20 gestational week using an endocervical sample collection device enhanced with surface nanostructures and/or microstructures, where the collection device enriches for fetal trophoblasts during the process of sample collection;
isolating the fetal trophoblast cells present in the endocervical sample;
conducting genetic testing of the isolated trophoblast cells.

19. The method of claim 18, wherein the surface of the endocervical sample collection device is further functionalized with fetal trophoblast specific antibodies.

20. The method of claim 18, where isolating the fetal trophoblast cells present in the endocervical sample comprises
treating the endocervical sample with a non-caustic mucolytic preparation to release intact and viable fetal trophoblast cells;
isolating individual viable fetal trophoblast cells using laser capture microdissection; and
culturing the individual fetal trophoblast cells to increase the genetic materials of the individual trophoblast cells.

21. The method of claim 20, wherein non-caustic mucolytic agent is selected from the group consisting of N-acetyl cysteine (NAC), ambroxol, sobrerol, carbocystein, carbocysteine sobrerol, letosteine, cithiolone, iodinated glycerol, N-isobutyrylcysteine, myrtol, erdosteine, dornase alfa, gelsolin, thymosin, dextra, and heparin.

22. The method of claim 18, wherein isolating the fetal trophoblast cells present in the endocervical sample comprises
isolating individual fetal trophoblast cells present in the collected endocervical sample by running the endocervical sample through a polymer microchip.

23. The method of claim 21, further comprising
conducting fetal genetic screening based on the genetic testing result.

24. The method of claim 21, further comprising
predicting risk for genetic birth defects based the genetic testing result.

25. The method of claim 21, further comprising
predicting pregnancy risk based on the genetic testing result.

26. The method of claim 21, further comprising
conducting fetal genetic diagnosis based on the genetic testing result.
